(19) United States
(12) Patent Application Publication
(10) Pub. No.: US 2003/0187379 A1
(43) Pub. Date: Oct. 2, 2003

(54) Hemodialysis emergency-disengagement-device

(76) Inventor: JIAN-DONG SUN, SAN JOSE, CA (US)

Correspondence Address: JIAN-DONG SUN, 1649 MERRILL DR. # 3, San Jose, CA, 95124, US

(21) Appl. No.: 10/115,313

(22) Filed: Apr. 2, 2002

(51) Int. Cl. A61M 37/00; C02F 1/44

(52) U.S. Cl. 604/5.01; 210/646

(57) ABSTRACT

The HEDD consists of a compressible body—top half (11) with a guide groove (12) and a cutting blade (13), a compressible body—bottom half (18) with a guide pillar (17), and a pair of special clamps (14) which are located to the left side and the right side of the guide pillar between the two halves of the compressible body (11 and 18). Under external compression, the two halves of the compressible body (11 and 18) are squeezed together to compress the special clamps (14) along the direction of the guide pillar (17). After the special clamps (14) completely block the hemodialysis blood-tubes (16) and are locked up due to elasticity, the cutting-blade (13) moves further to cut the hemodialysis blood tubes (16). When the external compression is released, the special clamps (14) and the blood tubes (16) are released from the device. The patient is then completely disengaged from the dialysis machine.

A Hemodialysis Emergency-Disengagement-Device (HEDD) is an accessory to kidney-dialysis equipment to allow patients to disengage themselves from hemodialysis machine in case of emergency.

The idea and/or a real object of the HEDD has never been used in renal clinics and never appeared in any relevant document.

The HEDD enables the patients themselves, in an emergency, to simply and rapidly clamp and sever the hemodialysis blood tubing attaching them to the dialysis machines, to expedite their escape from any potential danger.

The HEDD consists of 3 parts:

1. Compressible body

2. Special clamps

3. Cutting-blade.

The compressible body, consisting of the top half and bottom half, are made of plastics through a process of plastic molding. The cutting-blade is manufactured from sharpening a stainless-steel leaf. Both of them use only existing well-established technologies and can be easily realized.

Normally, a patient is seated and connected to a hemodialysis machine by a pair of hemodialysis blood tubes, rendering them immobile. In the case of an emergency, patients will find it very difficult to rapidly escape a dangerous situation. Any uncontrolled disconnection from the apparatus could lead to massive blood loss and possible death. At present, dialysis clinics handle patients in emergency situations (such as fires, earthquakes or terrorist attacks) as follows: in order to separate each patient from a dialysis machine, medical staff or patients must first find emergency kits, which usually hang on the dialysis machines or patient's chairs. Then medical staff or the patients have to use clamps to compress the venous and the arterial hemodialysis blood tubes, each of which needs two clamps to block blood flow. After that, they have to cut the venous-side and the arterial-side hemodialysis blood tubes between the clamps using scissors to disengage patients from the dialysis machines. Subsequently, the patients can evacuate the danger zone. This procedure requires a great deal of time. It is very likely that even with proper access to these emergency kits, many patients will not be able to skillfully use the clamps or scissors, especially in an emergency situation. Due to the nature of current hemodialysis methods, in reality a patient will only have one arm available to perform the entire procedure.

In most emergency situations, a matter of seconds could mean the difference between life and death. The purpose of the HEDD is to allow patients to very rapidly and easily disconnect themselves from hemodialysis machines in an emergency in case the need arises.

Before a patient undergoes dialysis, the staff or the patient can easily place each of the venous and the arterial hemodialysis blood tubes into two C-shaped notches of the special clamp in the device. The special clamps are located between the two halves of the compressible body, one on each side of the guide pillar. In emergency situations, the patient can squeeze the device with one hand. With the squeeze, the two halves of the compressible body move together along the guide pillar and the special clamps then block the hemodialysis blood tubes, effectively stopping the flow of blood. The cutting-blade then cuts both hemodialysis blood tubes at the end of the same squeeze. When the patient releases his hand, one set of clamped tubing disengages from the HEDD body. The patient then is free from the dialysis machine.

The following figures give a more detailed description of the device.

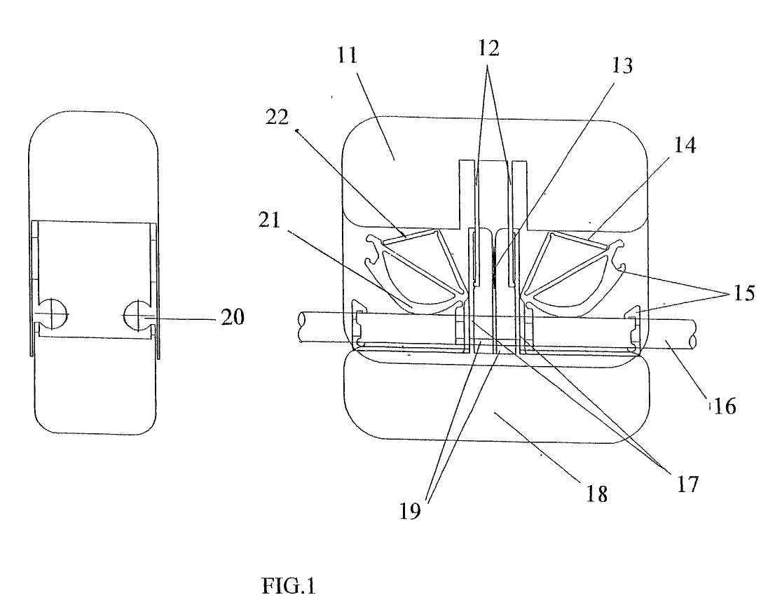

FIG. 1 is the assembly drawing.

11. Compressible body—top half

12. Guide groove

13. Cutting-blade

14. Special clamp with tubing closing structure

15. Locker

16. Hemodialysis blood tube

17. Guidepillar

18. Compressible body—bottom half

19. Pillow block

20. C-shaped notches of the special clamps through which the hemodialysis blood tubes pass

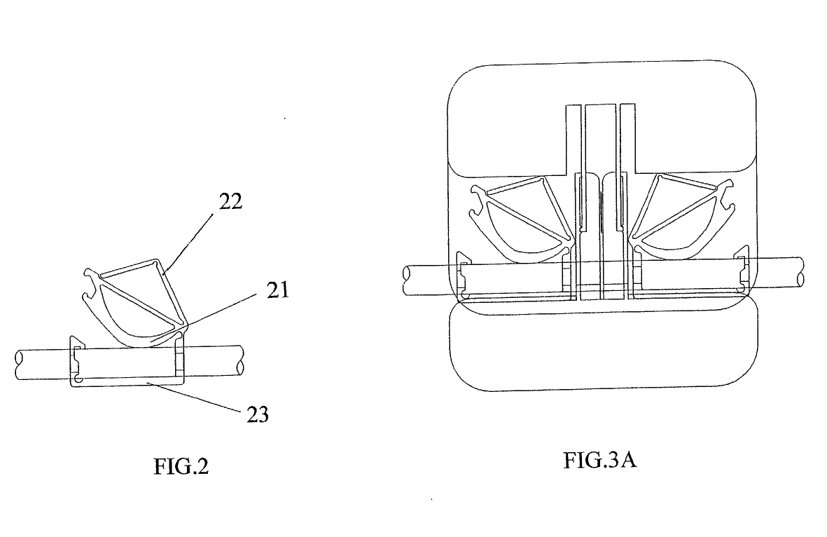

FIG. 2 shows the special clamp of the HEDD in detail

21. Bow-shaped head

22. Tubing closing structure

23. Base

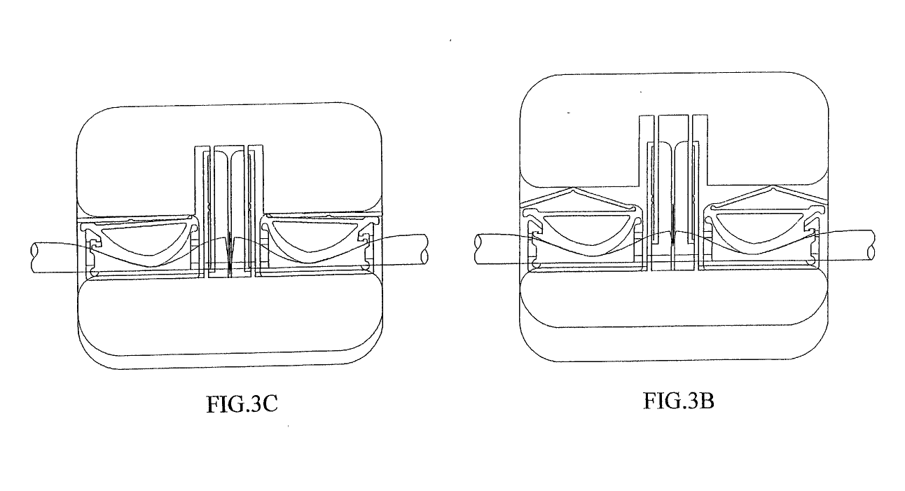

FIG. 3 shows the different states in operation of the HEDD. FIG. 3A Preparation state, FIG. 3B Compressed state at which the hemodialysis blood tubes are blocked, and FIG. 3C Final state when the tubing closing structure is pressed down completely and the special clamps are locked up then the cutting-blade cuts the tubes.

The detailed manual for using this product is as follows.

Insert the guide pillar 17 of the bottom half of the compressible body into the guide groove 12 of the top half of the compressible body. Place two special clamps 14 in it between the two halves of the compressible body, one on each side of the guide pillar, as shown in FIG. 3A. Put the hemodialysis blood tubes 16 into the C-shaped notches 20. When external compression is applied to the compressible body, the special clamps 14 are compressed and press on the hemodialysis blood tubes 16, while the bow-shaped head 21 presses against the base 23 to block the tubes and the lockers 15 are locked up due to elasticity, as shown in FIG. 3B. When the top half of the compressible body is pushed down further against the bottom half, the tubing closing structures 22 deform and flatten out and the cutting-blade 13 cuts into the pillow block 19 simultaneously, as shown in FIG. 3C. The hemodialysis blood tubes are now severed. Finally, when external force is released, the special clamps 14 attached to the severed blood tubing 16 disengage from the HEDD body.

This system holds numerous advantages over the current procedure. The device allows for extremely rapid disengagement from a hemodialysis machine with a simple squeeze with one hand on a device already attached to the blood tubing.

1. A hemodialysis emergency-disengagement-device, comprising:
a. a compressible body,
b. a framework within said device to enclose hemodialysis blood tubing,
c. at least one clamp within said device to permanently restrict the flow of any liquid upon compression of said device,
d. at least one cutting blade mounted within said device to sever said blood tubing,
e. means for controllably clamping and severing said blood tubing,
whereby said device will allow for rapid disengagement from hemodialysis machine upon external compression and release of said device.
